(19) United States
(12) Patent Application Publication
(10) Pub. No.: US 2008/0286736 A1
(43) Pub. Date: Nov. 20, 2008

(54) Orthopaedic Demonstration Aid

(76) Inventor: Oliver Browne-Wilkinson, London (GB)

Correspondence Address: YOUNG & THOMPSON, 209 Madison Street, Suite 500, ALEXANDRIA, VA, 22314, US

(21) Appl. No.: 11/664,824

(22) PCT Filed: Oct. 6, 2005
(86) PCT No.: PCT/GB05/50175

§ 371 Date: Apr. 6, 2007

(30) Foreign Application Priority Data
Oct. 7, 2004 (GB) 0422223.8
Dec. 9, 2004 (GB) 0426999.9
Jun. 17, 2005 (GB) 0512315.3

(51) Int. Cl. G09B 23/28; A61F 2/30

(52) U.S. Cl. 434/274; 623/18.11

(57) ABSTRACT

An orthopaedic skeletal joint demonstration aid for demonstrating surgical orthopaedic techniques comprises a first facsimile bone portion (2), a second facsimile bone portion (3), and adjustable holding mechanism (5) for holding the first facsimile bone portion (2) in a required anatomical orientation with respect to the second facsimile bone portion (3) for the purposes of a surgical orthopaedic procedure. The holding mechanism (5) is capable of being locked in position so as to orientate the first facsimile bone portion (2) at the required angle. This enables the facsimile bone portions (2) and (3) to be correctly aligned for carrying out a required surgical procedure, and for the alignment to be subsequently changed for a further surgical procedure.

This invention relates to orthopaedic demonstration aids, and is concerned more particularly, but not exclusively, with orthopaedic skeletal joint demonstration aids for demonstrating surgical joint replacement techniques.

Orthopaedic skeletal joint demonstration aids are known which consist of one or more facsimile human skeletal bones capable of demonstrating each stage of surgical alteration of the bone or bones in an orthopaedic technique to be demonstrated, including cutting of the bone and fitting of an orthopaedic prosthesis.

GB 2264190A discloses an orthopaedic demonstration aid in the form of a facsimile of the human skeletal knee with full femur and tibia, excluding patella, connected together in the correct anatomical position. Each of the femur and tibia comprises a main portion and a number of detachable pieces connected to the main portion by magnetic attraction. These pieces may be separately detached from the main portions to show different stages of an orthopaedic implant technique, and to accept the correct orthopaedic prosthesis. Once the demonstration has been completed, the prosthesis can be removed and the detachable pieces reattached so as to place the aid in its original state, suitable for reuse.

GB 2294573A discloses an orthopaedic demonstration aid in the form of a facsimile human skeletal bone having joint portions at opposite ends and an elongate hollow shaft interconnecting the joint portions and having a canal extending therealong. The facsimile human skeletal bone comprises two separable component parts, each consisting of a respective one of the joint portions and respective portions of the shaft and canal detachably connected together by a projection on one component part engaging within a receiving recess in the other component part in such a manner as to provide intercommunication between the canal portions within the two component parts.

WO04/064010 discloses an orthopaedic demonstration aid for demonstrating surgical orthopaedic techniques, the aid comprising first and second facsimile bone portions, an elongate first shaft portion to one end of which the first bone portion is secured, and adjustable holding means for holding a part of the first shaft portion remotely of said one end in a required position such that the first bone portion is at the correct anatomical orientation for the purposes of the required surgical orthopaedic procedure. In a preferred implementation the aid is an orthopaedic skeletal joint demonstration aid and the first and second facsimile bone portions are first and second joint portions capable of being manipulated relative to one another to mimic the action of a skeletal joint. However other implementations can also be envisaged in which the aid is to be used for demonstrating trauma, in which a bone is broken or shattered, and the first and second bone portions are two portions of a facsimile fractured bone which require to be set in required positions relative to one another. Such an orthopaedic skeletal joint demonstration aid may be used to demonstrate surgical orthopaedic techniques in joint replacement or modification, such as in hip, knee, shoulder, spine, digit, wrist, elbow and ankle replacement or modification, both for humans and animals.

It is an object of the invention to provide an improved orthopaedic demonstration aid which is capable of mimicking the natural kinematics of the bone in the required manner and which is particularly convenient in use.

The invention is defined by the accompanying claims.

In order that the invention may be more fully understood, a preferred embodiments of orthopaedic skeletal joint demonstration aid in accordance with the invention will now be described, by way of example, with reference to the accompanying drawings, in which:

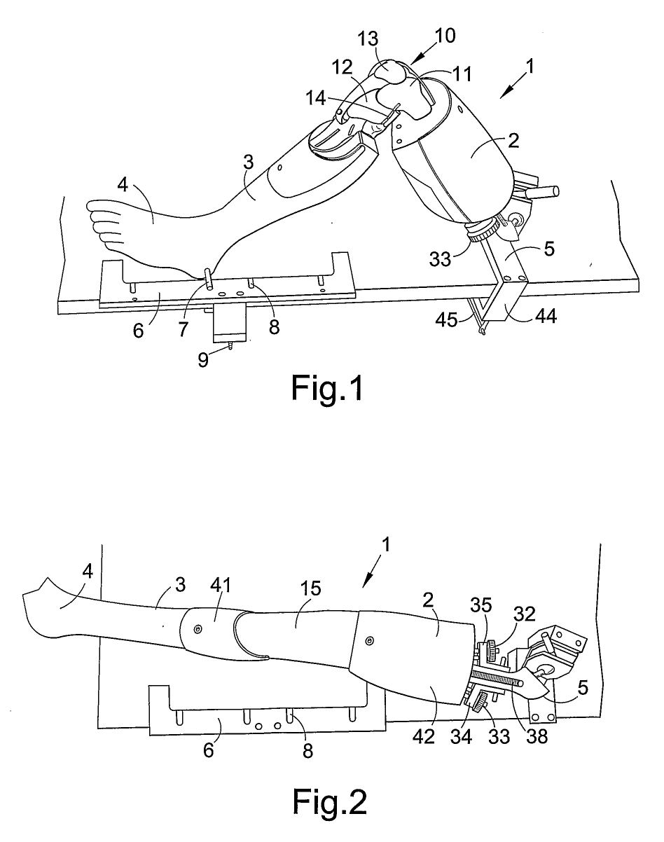

FIG. 1 is a perspective view of a first embodiment;

FIG. 2 is a view from above of the first embodiment;

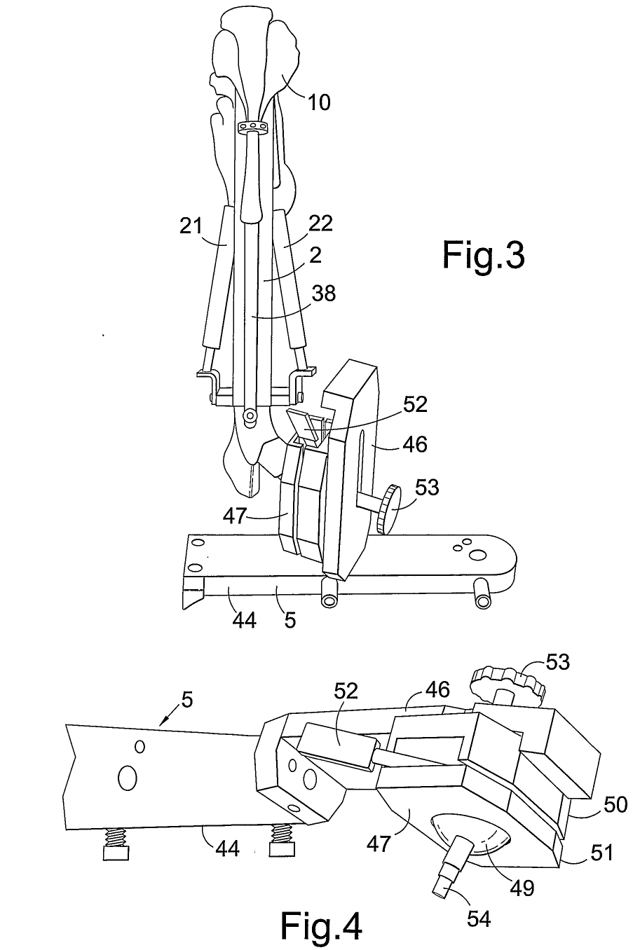

FIG. 3 is a view from behind of a second embodiment;

FIG. 4 is a view of a holding mechanism of the second embodiment;

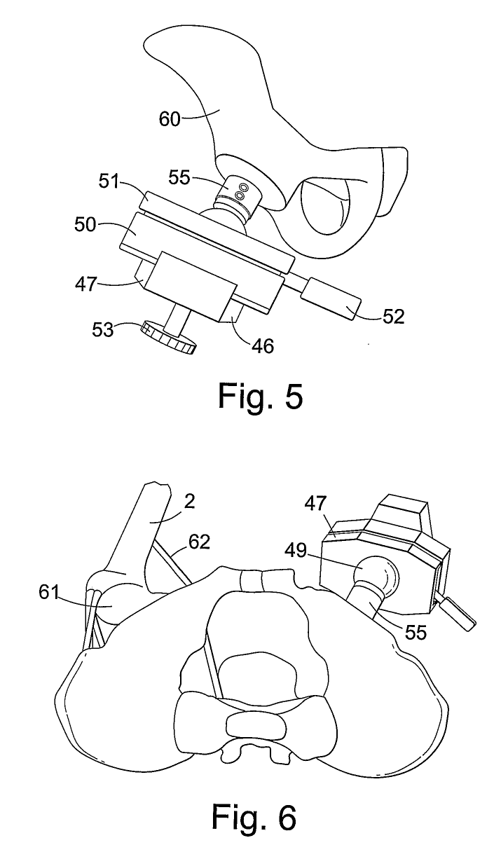

FIG. 5 shows the attachment of a facsimile pelvis to the holding mechanism of a third embodiment;

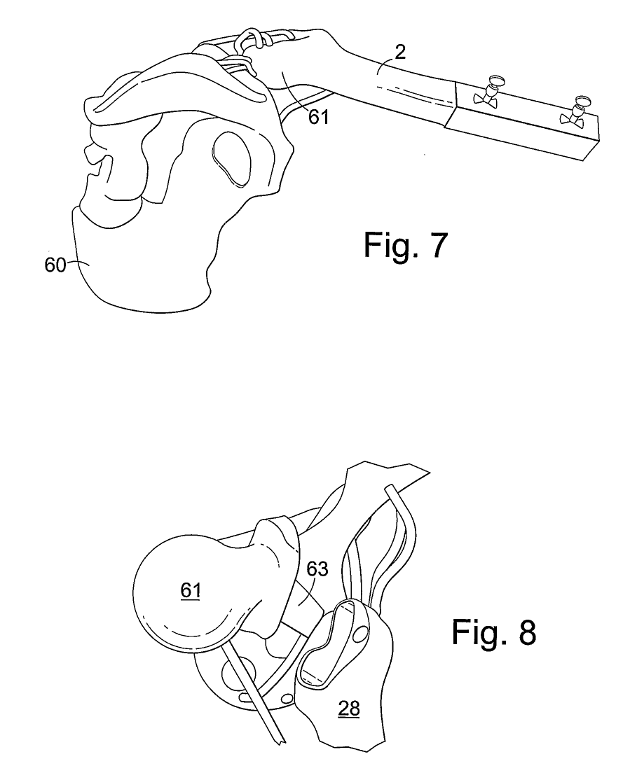

FIGS. 6 and 7 are perspective views of successive positions in the dislocation of a facsimile leg from the facsimile pelvis of the third embodiment;

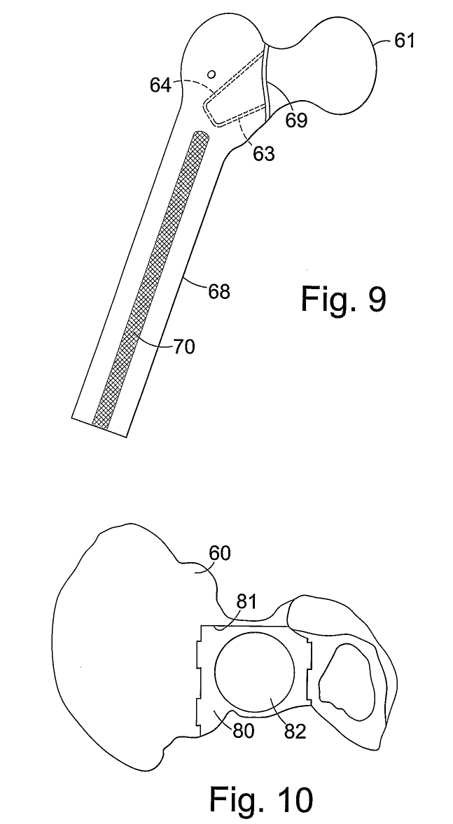

FIG. 8 is a perspective view of a detachable joint part of a facsimile femur of the third embodiment, FIG. 9 showing a diagrammatic view of a variant of the detachable joint;

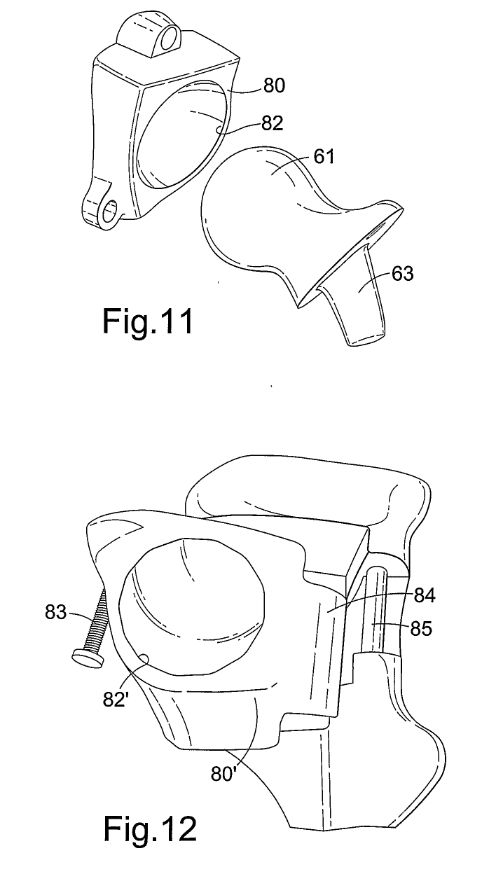

FIGS. 10 and 11 are perspective and diagrammatic views of a detachable socket part of a facsimile pelvis of the third embodiment;

FIG. 12 is a perspective view of a detachable socket part of a facsimile pelvis of a fourth embodiment;

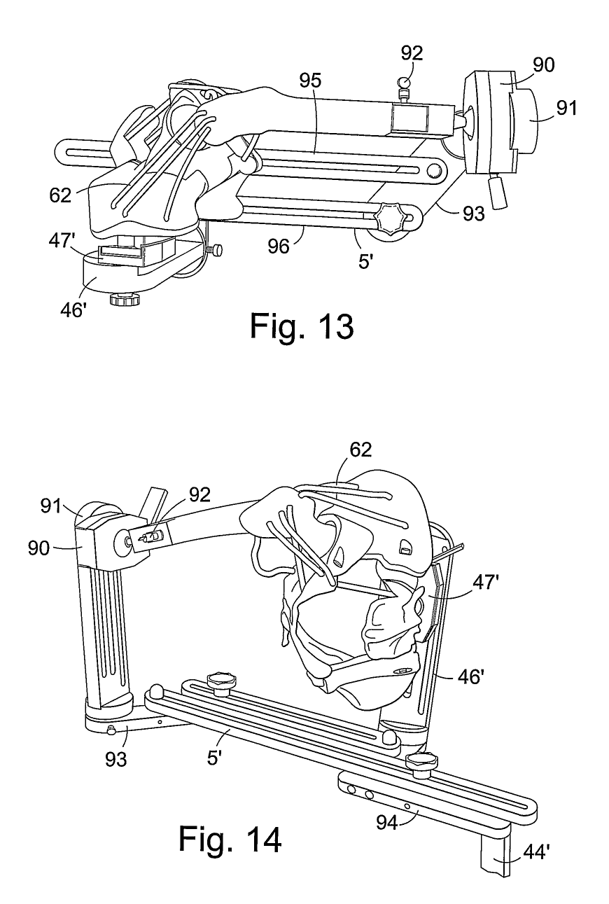

FIGS. 13 and 14 are perspective and diagrammatic views of a fifth embodiment;

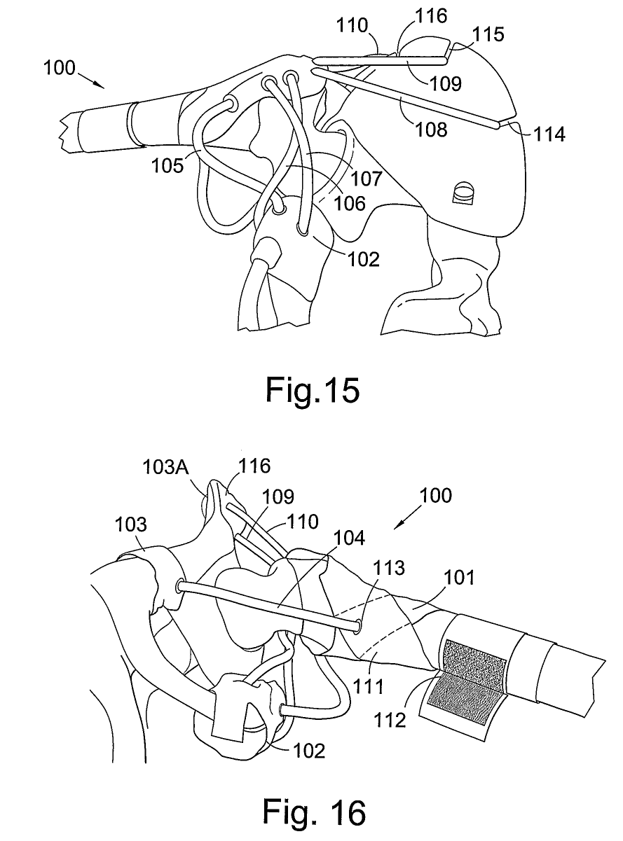

FIGS. 15, 16 and 17 are perspective views of a sixth embodiment; and

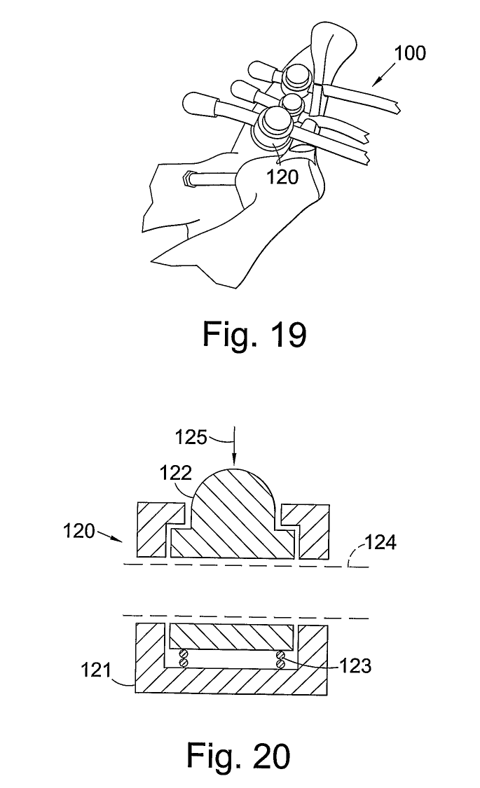

FIGS. 18 and 19 are perspective views of a seventh embodiment, FIG. 20 being a section view through one of the toggles used in this embodiment.

Referring to FIG. 1 the demonstration aid 1 shown therein comprises a facsimile human thigh portion 2, a facsimile human calf portion 3 and a facsimile human foot portion 4, as well as a holding mechanism 5 and a foot plate 6 provided for holding the thigh portion 2 and the calf portion 3 in the required relative positions mimicking the correct anatomical orientation for the purposes of the required surgical orthopaedic procedure. As will be described more fully below the holding mechanism 5 is capable of being locked in position so as to orientate the thigh portion 2 at the required angle, and the foot portion 4 is provided with two outwardly extending pegs 7 (one projecting from each of the sides of the heel) capable of being engaged in one of a series of receiving notches 8 in the foot plate 6 to orientate the calf portion 3 at the required angle (each notch corresponding to a different angle, e.g. 90 degrees, 110 degrees, 125 degrees, etc.). The foot plate 6 is provided with a screw clamp 9 by means of which it may be detachably secured to an operating surface. Although not shown in the figures, the foot plate 6 is connected to the holding mechanism 5 by a predetermined length of cord such that the foot plate 6 is spaced from the holding mechanism 5 by the required distance if the foot plate 6 is positioned such that the cord is taut prior to fixing of the foot plate 6. This ensures that the calf portion 3 is held at the required angles when engaged with the receiving notches 8. The aid 1 also comprises a facsimile knee joint 10 consisting of a first (femur) joint portion 11, a second (tibia) joint portion 12, a facsimile patella portion 13 and a facsimile ligament portion 14.

FIG. 2 shows the aid 1 from above with the foot portion 4 detached from the foot plate 6, and with a detachable facsimile soft tissue (or skin) portion 15 in place so as to cover the knee joint. The facsimile soft tissue portion 15 is made of an inscissable silicone material mimicking the properties of human soft tissue and can be left in position during a surgical procedure, including cutting such as to cut the soft tissue portion 15. In a variant embodiment the soft tissue portion may be precut at the location in which surgical cutting is required.

The first and second joint portions 11, 12 of the knee portion 10 mimic the form of the joints of the human femur and tibia and have square-section connecting parts and adapted to detachably engage within receiving square-section passages in aluminium box-section shaft members of the thigh portion 2 and the calf portion 3. Each of the thigh portion 2 and the calf portion 3 is made up of several premoulded parts indicative of the soft tissue (or subdermal layer) that are detachably connected to the associated shaft member. Furthermore the facsimile ligament portion 14 is in the form of a continuous loop of cord passing through a cross-passage in the second joint portion 12 and two symmetric passages in the first joint portion 11 such that the two ends of the ligament portion 14 pass through a central canal within the connector to enable them to be connected to a tensioning arrangement after passing through a canal within the shaft member of the thigh portion. The patella portion 13 is held in place by a further ligament portion in the form of a closed loop of cord passing through symmetric passages in the patella portion and having its ends glued within passages in a tibial tuberosity of the second joint portion 12.

The manner in which the ligament portions are tensioned will now be briefly described. The second joint portion 11 is detachably connected to the thigh portion 2 by the connector part engaging within the square-section shaft member of the thigh portion. The shaft member is provided with side windows by means of which the ends of the ligament portion 14 may pass laterally out of the shaft member to engage with the tensioning mechanism comprising two adjusting screws 32 and 33 having milled adjusting heads and extending through two angled flanges 34 and 35 (to permit proper drawing of the ligament portion ends) connected to the shaft member. The ligament portion ends are provided with ferrules which are detachably connectable to receiving parts on the ends of the screws 32 and 33. The receiving parts are maintained under spring tension so as to enable each ferrule to be hooked into the appropriate part from the side with the part compressed against the spring force and for the ferrule to be positively engaged to tension the ligament portion 14 after the part has been released.

The tensioning screws 32 and 33 may be turned to adjust the tension applied to each of the ligament portion ends so that the tension of each part can be independently adjusted to maintain the knee joint perfectly balanced with the same gap on either side of the joint both when the knee is in flexion and in extension (straight). The trimming of the knee ligaments (by partial cutting) in a surgical procedure may therefore be simulated by appropriate adjustment of the tensioning screws 32 and 33. It will be appreciated that the connection of the ligament portion ends to the tensioning screws 32 and 33 provides a quick-release mechanism by which the ligament portion 14 may be released to enable the knee joint to be removed from the aid after completion of a surgical procedure and replaced by a fresh knee joint. It is also important that the tensioning should allow sufficient resilience to permit a small amount of medial and lateral flexing of the knee joint. In an alternative arrangement the ligament portion ends are connected to the tensioning screws 32 and 33 by turnbuckle connectors which are adjustable by turning of the turnbuckle connectors. In this case the resilience is imparted to the ligament portion by separate springs provided at ligament portion ends.

A spring 38 is provided for tensioning the ligament portion in the correct way for positioning of the patella 13, although ligament portion is not shown connected to this spring 38 in the figures. Although not shown in the figures, the free loop of the ligament portion passes through a passage in the thigh portion and is engaged with the end of the spring 38 so as to provide the correct anatomical positioning of the patella portion 13 in relation to the femur and the tibia.

The calf portion 3 includes connecting nipples symmetrically positioned on either side of the calf portion 3 for engaging within corresponding recesses in the soft tissue portion 15 (see FIG. 2) so as to securely hold the soft tissue portion 15 in position by means of a cover portion 41 screwed to the calf portion 3 as shown in FIG. 2. The soft tissue portion 15 is thereby securely attached to the calf portion 3 by being trapped between the cover portion 41 and the calf portion 3. Although not shown in the figures a similar arrangement may be utilised for connecting the soft tissue portion 15 to the thigh portion 2, the detachable cover being shown at 42 in FIG. 2. These connection arrangements enable the soft tissue portion 15 to be held under tension over the knee joint so as to mimic the flexing of the skin as the knee joint is bent.

As may be seen in FIG. 1, the holding mechanism 5 incorporates a screw clamp 44 for detachably attaching the holding mechanism 5 to an operating surface. FIG. 1 also shows the adjusting screw 45 by means of which the clamp 44 may be secured to the operating surface in a known manner. As best seen in FIG. 5 the holding mechanism 5 also includes a locking assembly 47 connected on one side to a plate 46 and connectable on the other side to a hip portion (not shown in FIG. 5). The connection of the plate 46 to the hip portion is by means of a stub axle 54 connected to a ball 49 held within a receiving socket between two plates 50 and 51 that are capable of being moved together and apart by a locking lever 52. When the locking lever 52 is in a released position the plates 50 and 51 are held apart sufficiently to enable the ball 49 to be rotated within the socket to permit adjustment of the position of the hip portion. However, when the locking lever 52 is moved into in a locking position, the plates 50 and 51 are compressed against the ball 49 in such a manner as to prevent the ball 49 from being rotated within the socket so that the hip portion is locked in position. The hip portion is connected to the ball 49 at a compound angle such that the thigh portion is held at the correct anatomical angle in two mutually perpendicular planes, that is at approximately 135 degrees on one plane and at approximately 15 degrees in the other plane relative to the pelvis. This ensures the correct alignment in the aid of the anatomical axes of both the femur and the tibia relative to the mechanical axis passing through the hip and knee joints. Height adjustment is provided by an adjusting screw 53 extending through a slot in the plate 46 and capable of being tightened to hold the locking mechanism 47 at a defined position with respect to the plate 46.

FIG. 3 shows a second embodiment in which the adjusting screws of the tensioning mechanism are replaced by ferrules on the ends of 21 and 22 of the ligament portion 14. In these two views the thigh and calf portions 2 and 3 are shown without their premolded soft tissue parts.

FIGS. 5, 6 and 7 illustrate the manner of attachment of a pelvis portion 60 in a third embodiment (in which the arrangement is connected to a pelvis portion rather than to a calf portion as in the previously described embodiments) by insertion of the stub axle 54 of the locking assembly 47 into a quick release connector 55 within which the stub axle 54. For release of the stub axle 54 from the connector 55, the connector 55 is preferably rotated through a limited angle. Although the connection is made to the right hand side of the pelvis portion in this embodiment, the connection is made to other parts of the pelvis portion, for example the sacrum, in other embodiments of the invention.

In this third embodiment the thigh portion 2 is provided with a joint part 61 for engaging within a socket part in the pelvis portion 60, the joint part 61 being held within the socket part by an elastic cord 62 denoting a muscle attachment.

The elasticity of the cord 62 enables the joint part 61 of the thigh portion 2 (the femur) to be manipulated within the socket part (acetabulum insert) of the pelvis portion, so as to dislocate the thigh portion 2 and enable it to be moved into its correct anatomical position for the performance of a surgical procedure in which the acetabulum cup is revealed so that it can be machined as required. Similarly the joint part 61 may be machined when the thigh portion is held in this position. Once both procedures have been completed, and the required prosthesis fitted, the joint part 61 can be articulated relative to the socket part so as to place it back in its original position. This therefore enables a simulation of the total hip replacement procedure.

To enable the joint part 61 to be replaced after machining in such a procedure, so as to enable the demonstration aid to be used in a subsequent surgical procedure, joint part 61 is preferably detachable as shown in FIG. 8. FIG. 8 shows an arrangement in which the joint part 61 is separable from a main part 68, the joint part 61 being provided with a tapered projecting portion 63 which is an interference fit within a corresponding tapered receiving recess 64 in the main part 68. The shape of the projecting portion 63 and the receiving recess 64 is such as to provide an anti-rotation pinch locking mechanism that prevents the joint part 61 from being rotated about the axis of the main part 68, and prevents migration of the joint part 61 during the surgical procedure. Disposable joint part 61 is made from a material that allows for milling, reaming, drilling, nibbling and broaching of the part to allow for all types of resurfacing, primary and minimal invasive surgical procedures. It is also possible to provide a facsimile soft tissue attachment to the joint part 61 and the main part 68 which may itself be detachably connectable to the shaft member.

FIG. 9 shows a variation of this arrangement in which like parts are denoted by the same reference numerals as in FIG. 8. In this variant the connecting line 69 between the joint part 61 and the main part 68 is somewhat closer to the extremity of the joint part 61, and a hollow canal 70 is provided within the main part 68. If required the socket part 61 may be held in engagement with the main part 68 by magnetic attraction of embedded magnets within these parts.

FIGS. 10 and 11 show a detachable socket part 80 that is detachably receivable within a receiving recess 81 in the pelvis portion 60. The socket part 80 defines an acetabulum cup 82 within which the joint part 61 representing the head of the femur is engageable. The socket part 80 is held within the receiving recess 81 as an interference fit by means of an anti-rotation pinch lock mechanism that stops the socket part from being rotated around its anatomical axis in relation to the pelvis portion 60 and prevents migration of the socket part 80 during a surgical procedure. Other means of locking the socket part within the receiving recess can alternatively be used. If required the socket part 80 may be held in place within the receiving recess 81 by means of the magnetic attraction of embedded magnets. In an alternative embodiment shown in FIG. 12 the socket part 80′ defining the acetabulum cup 82′ is held in place by a hook part 84 engaging a shaft portion 85 of the pelvis portion 60 and a screw 83 passing through the socket part 80′ into a screwthreaded hole in the pelvis portion 60. The socket part 80 or 80′ is made from a material that allows for milling, reaming, drilling, nibbling and broaching of the part to allow for all types of resurfacing, primary and minimal invasive surgical procedures. If required provision may be made for soft tissue attachment to the socket part and the connecting portion of the pelvis.

As shown in the figures a detachable socket part 80 is provided on only one side of the pelvis portion 60, the other side of the pelvis portion serving for detachable connection to the stub axle 53 of the locking assembly 47. However, it is also possible for both the left and right sides of the pelvis portion to be provided with disposable socket parts defining acetabulum cups.

Each of the detachable joint part and the detachable socket part arrangements described above may be used on their own or together with one another to demonstrate different surgical procedures. The joint part can be machined in many different ways so as to accept a variety of different hip implant prostheses during surgical procedures, and can then be disposed of and replaced by a fresh joint part so that the aid can be used for demonstration of a further surgical procedure. If required the fresh joint part can be a joint part that is distorted to simulate a deformity. Furthermore the socket part can be machined in many different ways so as to accept a variety of different acetabular implant prostheses in different surgical procedures, and the socket part may be replaced by a fresh socket part as required to enable the aid to be used for demonstration of a new surgical procedure. The socket part is machinable to receive either the joint part or the corresponding part of a prosthesis that is fitted after machining of the joint part to enable fitting of the prosthesis. The socket part is preferably provided with anatomical features that align to the external form of the facsimile pelvis portion. The joint part preferably also has an anatomical head that aligns to the external form of the main part of the facsimile femur.

It is important to appreciate that each of the joint part and socket part arrangements may be used individually as demonstration aids for demonstrating a particular aspect of orthopaedic hip replacement on one side of the joint, with the disposable part in each case being simply the joint part or the socket part rather than the whole of the facsimile bone. Furthermore the facsimile proximal femur may be connected in the correct anatomical relationship to a constant distal femur shaft which may in turn be connected to a distal femur with the correct anatomical relationship, resulting in a full femur bone, as described above in relation to the knee arrangement. Furthermore the socket part may be connectable to a facsimile hemi pelvis that may in turn be connectable to a full facsimile pelvis.

The orthopaedic skeletal joint demonstration aid described above with reference to the FIGS. 1 to 12 of the drawings is a modular joint replacement simulator for interchangeably holding (i) a facsimile human leg having portions mimicking the human knee joint and portions mimicking the form of the human thigh, calf and foot, and (ii) a facsimile human pelvis in the correct anatomical positions. However, the invention is also applicable to a wide range of other forms of orthopaedic skeletal joint demonstration aid, including aids incorporating only portions mimicking other parts of the skeleton, including the hip, elbow, shoulder, spine and other joints.

The modular joint replacement demonstration aid allows for the anatomical orientation of one or more human joint portions to be orientated into lockable positions prior to orthopaedic surgery. It also allows for incorrect orientation of the joint portions to simulate the problems that may occur if the patient was positioned incorrectly.

The demonstration aid is initially releasably secured to a work surface, the required joint portion(s) being then attached by means of a snap or other connection. Alternatively the joint portion(s) may be permanently attached to the pelvis portion. The positioning of the portion(s) can be fine tuned using the orientating holding mechanism that permits changes in height and orientation to give the desired surgical position of the joint prior to orthopaedic joint replacement surgery.

The demonstration aid has a base clamp for securing the aid to a work surface, and the holding mechanism is releasably secured to the base clamp. The holding mechanism is positioned on the base clamp to give a general working space around each joint portion. The height of the holding mechanism is then adjusted for each joint portion to simulate its correct position relative to an operating table. The rotational positioning of the joint portion is required to simulate the complex angles of the joint portion relative to an operating table and relative to other parts of the human anatomy. The combination of these three elements allows for the joint replacement demonstration aid to exactly align the joint portion prior to a joint replacement simulation workshop taking place. The height adjustment on the holding mechanism is controlled via a through channel and pinch wheel. The orientation of the joint area is controlled via a releasable ball and socket joint that is part of the holding mechanism. By releasing the ball joint, the joint portion is free to rotate about all axes and can then be locked in the required position for simulation.

A joint portion is connected to the holding mechanism by a quick release mechanism that has the female connector housed in the joint portion and the male connector attached to the ball of the holding mechanism. Each joint portion can be easily released from the holding mechanism, so as to enable a new or different joint portion to be fitted.

A further embodiment of the invention will now be described with reference to FIGS. 13 and 14 of the accompanying drawings. This embodiment is again a modular joint replacement simulator for interchangeably holding (i) a facsimile human leg having portions mimicking the human knee joint and portions mimicking the form of the human thigh, calf and foot, and (ii) a facsimile human pelvis in the correct anatomical positions, although the portions mimicking the human knee joint, calf and foot are in this case detachable and are not shown in place in these figures (although they generally have the form shown in the other figures).

In this embodiment, as best seen in FIG. 14, the holding mechanism 5′ (which is of a completely different form to the holding mechanism 5 already described) incorporates a screw clamp 44′ for detachably attaching the holding mechanism 5′ to an operating surface. The holding mechanism 5′ also includes a locking assembly 47′ connected on one side to a plate 46′ and connectable on the other side to the sacrum of the pelvis portion (and not to the right hand hip portion as in the previously described embodiments) by means of a stub axle connected to a ball held within a receiving socket between two plates that are capable of being moved together and apart by a locking lever as in the previously described embodiments. Height adjustment is again provided by an adjusting screw extending through a slot in the plate 46′ and capable of being tightened to hold the locking mechanism 47′ at a defined position with respect to the plate 46′.

By contrast with the previously described embodiments, however, the holding mechanism 5′ also includes a locking assembly 90 (of similar form to the locking assembly 47′) connected on one side to a plate 91 and connectable on the other side to the proximal femur by a clamping screw 92 extending through a connector part connected to a ball held within a receiving socket of the locking assembly 90. As with the locking assembly 47′, the locking assembly 90 is capable of being released by adjustment of the locking lever to enable the proximal femur to be moved not any required position, as well as being adjustable in height by an adjusting screw extending through a slot in the plate 91. The holding mechanism 5′ further includes a twin-bar system interconnecting the plates 46′ and 91 and comprising two support plates 93 and 94 on which the plates 46′ and 91 are rotatably supported (and lockable in required rotary positions by means of locking screws) and two parallel bars 95 and 96 interconnecting the support plates 93 and 94. Each of the parallel bars 95 and 96 is pivotally connected at one end to a respective one of the support plates 93 and 94, and is pivotally connected to the other plate 93 or 94 by a locking knob 97 or 98 capable of being tightened to hold the locking assemblies 47′ and 90 in the required positions relative to one another.

It will be appreciated that the degree of adjustment permitted by the holding mechanism 5′ is such that the proximal femur may be held in virtually any orientation relative to the pelvis that may be required for a particular surgical procedure. A comparison of FIGS. 13 and 14 gives an indication of the very considerable degree of adjustment, including adjustments in X, Y and Z (height) directions and rotation (in particular rotation of the femur about its axis permitted by the ball of the locking mechanism 90), permitting by such a mechanism. If required for demonstration of a different procedure, the proximal femur may be released from its attachment to the connector part of the locking mechanism 90 after loosening of the clamping screw 92, and attached to the portions mimicking the form of the human thigh, calf and foot to provide a complete assembly broadly similar to that shown in FIGS. 1 to 3.

In each of FIGS. 13 and 14 cords 62 denoting the muscle attachments are shown interconnecting the joint part of the femur and the pelvis portion, these being detachable to enable the femur to be dislocated and moved into its correct anatomical position for the performance of a surgical procedure in which the acetabulum cup is revealed so that it can be machined as required. Once the procedure has been completed, and the required prosthesis fitted, the joint part can be articulated relative to the socket part so as to place it back in its original position. The cords 62 are permanently attached to the main part 68 of the femur, the free ends of the cords 62 being detachably attachable to the pelvis by releasable fittings (not shown in FIGS. 13 and 14) on the pelvis. Alternatively the cords 62 may be permanently attached to the pelvis, and free ends of the cords 62 may be detachably attachable to the main part 68 of the femur by releasable fittings. Optionally the detachable joint part 61 is attached to the main part 68 by a screw 99 extending through the joint part 61 into a screwthreaded hole in the main part 68, rather than the connection simply relying on the interference fit of the tapered projecting portion 63 within a corresponding tapered receiving recess in the main part 68.

The illustrated embodiment incorporates a full hip and left leg to knee as one joint portion and the full lower leg as a second joint portion. Other human joint portions, such as the shoulder, spine, elbow, ankle, digit and wrist could also be used.

In the case of a demonstration aid for the knee, for example, the knee may be connected via a quick release mechanism to the ball of the locking clamp which has a variable height mechanism built into it. The ball represents the head of the femur and the connector the neck of femur, thus allowing the rest of the model to be anatomically correct in relation to the clamp.

In the case of a demonstration aid for the hip, a total pelvis is provided with a connector located in the right hand side cup for attaching the total pelvis to the ball and socket clamp. By releasing the ball the pelvis can be rotated into a series of different positions and secured allowing the pelvis to be worked on to simulate various orthopaedic procedures, such as hip replacement for example.

In the case of a demonstration aid for the spine, the arrangement should be such that the pelvis can be released and the clamp head moved to a new orientation. Once the head is locked into its new position the spine model can then be attached and orientated into its correct anatomical position to simulate spine surgery.

In the case of a demonstration aid for the shoulder, the previously described model could be released, the clamp head being then moved to the correct anatomical position for the shoulder and a scapula with a connector attached. Again the model could be orientated into is correct anatomical position to simulate shoulder surgery. Similar models can be applied to other joints of the body.

A further embodiment of the invention will now be described with reference to FIGS. 15, 16 and 17 which is a variant of the embodiment of FIGS. 13 and 14 in which the permanently attached cords 62 denoting the muscle attachments are replaced by a detachable ligament arrangement 100 interconnecting the joint part of the femur and the pelvis portion. The ligament arrangement 100 is such as to enable the femur to be dislocated and moved into its correct anatomical position for the performance of a surgical procedure in which the acetabulum cup is revealed so that it can be machined as required, without any parts of the ligament arrangement 100 having to be detached from the femur and the pelvis portion.

The ligament arrangement 100 comprises four sling parts 101, 102, 103 and 103A in the form of shaped strips of fabric interconnected by four elastic cord parts 104, 105, 106 and 107, and three further elastic cord parts 108, 109 and 110. Although seven cord parts are shown in total in the illustrated embodiment, the number of cord parts may be varied depending on the level of sophistication of the aid, and other embodiments are also possible in which there are thirteen or seventeen cord parts in total in order to represent the action of the different muscle groups. In a further variant the ligament arrangement is not such as to enable the femur to be dislocated in that the cord parts are inelastic, the cord parts being adapted to be detachable to simulate the action of detachment of one or more muscle groups in a surgical procedure.

In the illustrated embodiment the sling parts 101, 102, 103 and 103A are detachably connectable to parts of the femur and the pelvis by being wrapped around the appropriate part and are capable of being secured thereon by the attachment of the hooks and eyes of Velcro strips 112 on the sling parts to one another, as best seen in FIG. 16. If required one or more Velcro pads may also be provided on the interior faces of one or more of the sling parts for detachable engagement with corresponding Velcro pads on the femur or pelvis with the object of correctly aligning the sling part on the bone. In a development of the invention the cord parts are adjustable in length, for example by having adjustable buckles, so as to enable the cord parts to be set to required tensions according to the required overall relationship of the femur to the pelvis. For example the cord part lengths may be set to simulate different sorts of muscle wastage or skeletal deformity that it is intended to simulate for the purpose of demonstration of surgical techniques.

More particularly the ligament arrangement 100 comprises a sling part 101 that is sewn to itself in a cross-over region 111 to form a sleeve through which the femur can be passed before the sling part is secured in position by the Velcro strips 112. A further sling part 102 is attachable to a part of the pelvis by again being wrapped around that part and secured in position by Velcro strips. A further sling part 103 is attachable to another part of the pelvis by again being wrapped around that part and secured in position by Velcro strips. In each case the cord parts extend through reinforced holes 113 in the sling parts and are held therein by being knotted (or provided with a ferrule) within the sling part or by re-emerging from another reinforced hole in the case where two or more of the cord parts are integral parts of the same length of cord. The elasticity of the cord parts enables mimicking of the dislocation of the joint as previously described.

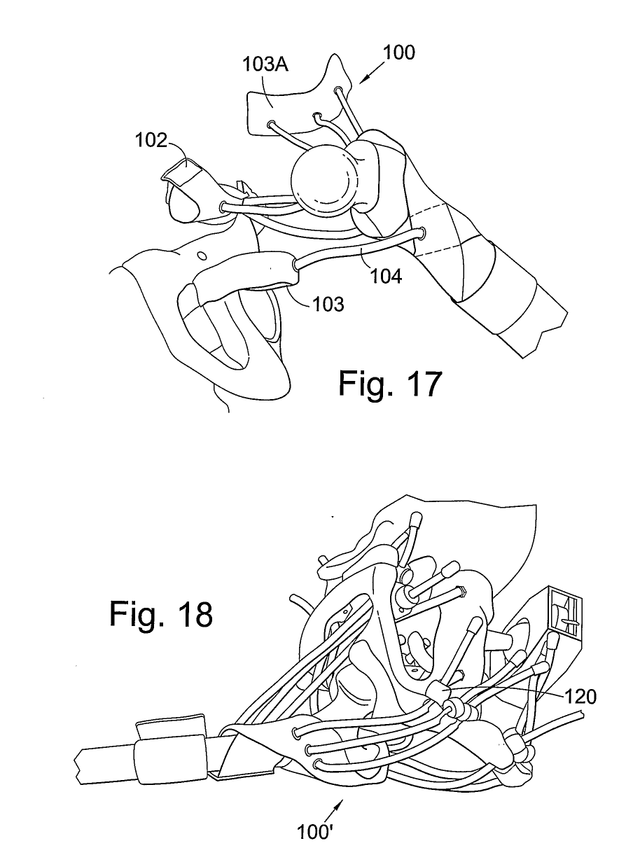

Additionally the ligament arrangement 100 comprises a further sling part 103A to which the cord parts 108, 109 and 110 are attached and that serves to secure the three cord parts 108, 109 and 110 together in such a manner as to permit the cord parts 108, 109 and 110 to be received within three slots 114, 115 and 116 in the pelvis when the sling part 103A is positioned behind the pelvis as shown in FIG. 15. FIG. 17 shows the manner in which the sling parts 101, 102, 103 and 103A can be detached from the pelvis and the femur to enable replacement of any part of the pelvis or femur or any of the sling or cord parts that requires replacement.

In a further possible ligament arrangement 100′ shown in FIGS. 18 and 19, the arrangement is generally similar to the arrangement 100 of FIGS. 15, 16 and 17 but with the cord parts being provided with spring toggles 120 which hold the cord parts in the slots within which they are received in the pelvis and which are adjustable in their positions along the cord parts to vary the length of each cord part interconnecting the pelvis and the femur, for example. FIG. 20 is a section through a spring toggle 120 comprising an inner button 122 displaceable within an outer casing 121 against a compression spring 123 by the action of a compressive force applied in the direction of the arrow 125, in order to release the toggle 120 from a cord part 124 (shown in broken lines) extending through aligned holes in the inner button 122 and the outer casing 121 so that the toggle can be moved along the cord part 124. As best seen in FIG. 19 the position of the toggle 120 on the cord part can be appropriately adjusted in this manner and, after release of the compressive force on the button to permit the toggle to become clamped to the cord part, the cord part may then be introduced into the slot in the pelvis with the toggle 120 engaging behind the slot to secure the cord part in position.

It will be appreciated that various modifications of the above-described demonstration aids are possible to adapt them for demonstration of particular surgical orthopaedic techniques, and, in each case, the aid may be in the form of a facsimile of one or more bones or a portion of one or more bones. For example, in addition to the arrangement described above for tensioning the facsimile collateral ligaments, a similar but separate arrangement may be provided for tensioning facsimile cruciate ligaments provided on the inside of the knee joint. The aid may also be used to demonstrate other types of surgical technique.

1-36. (canceled)

37. An orthopaedic demonstration aid for demonstrating surgical orthopaedic techniques, the aid comprising adjustable holding means for holding at least one facsimile bone portion in a required anatomical orientation for the purposes of a surgical orthopaedic procedure.

38. An aid as claimed in claim 37, wherein the holding means is arranged to hold first and second facsimile bone portions in such a manner as to render them capable of being manipulated relative to one another to mimic the action of a skeletal joint.

39. An aid as claimed in claim 37, wherein the holding means comprises an adjustable ball and socket joint and a locking mechanism for locking the ball and socket joint when in the required position.

40. An aid as claimed in claim 37, wherein the holding means incorporates releasable securing means for holding said at least one facsimile bone portion in such a manner that said at least one facsimile bone portion is readily detachable to enable replacement of said at least one facsimile bone portion by a replacement facsimile bone portion for a new surgical orthopaedic procedure.

41. An aid as claimed in claim 37, wherein the holding means is adjustable to vary the height at which said at least one facsimile bone portion is supported.

42. An aid as claimed in claim 37, wherein the holding means is adjustable to vary the orientation of said at least one facsimile bone portion.

43. An aid as claimed in claim 37, wherein the holding means incorporates a plate for releasable attachment to a fixed support and detachable connection means for connecting said at least one facsimile bone portion to the plate, the detachable connection means comprising a plurality of connection points spaced apart on the plate to which said part of the second shaft portion is selectively connectable.

44. An orthopaedic skeletal joint demonstration aid for demonstrating surgical orthopaedic techniques, the aid comprising a first facsimile bone portion, a second facsimile bone portion, and releasable holding means for interchangeably holding the first facsimile bone portion and the second facsimile bone portion in their required anatomical orientations for the purposes of different surgical orthopaedic procedures.

45. An orthopaedic skeletal joint demonstration aid for demonstrating surgical orthopaedic techniques, the aid comprising a facsimile skeletal bone including an elongate main part having an enlarged portion at one end, and a detachable joint part for attachment to the enlarged portion by means of projecting portion of one of the parts that extends into a receiving recess in the other of the parts, whereby, after a surgical procedure has been carried out on the joint part while attached to the main part, the joint part may be detached from the main part and replaced by a fresh joint part.

46. An aid as claimed in claim 45, wherein the joint part is made from a material that allows for milling, reaming, drilling, nibbling or broaching of the joint part to allow for a resurfacing, primary or minimal invasive surgical procedure.

47. An aid as claimed in claim 45, wherein the joint part is detachably attached to the enlarged portion by means of a projecting portion of the joint part that extends into a receiving recess in the enlarged portion

48. An aid as claimed in claim 45, wherein the joint part is detachably attached to the enlarged portion by means of an interference locking fit or a connecting screw.

49. An aid as claimed in claim 45, wherein the joint part is detachably attached to the enlarged portion by means of an arrangement that prevents relative rotation between the joint part and the enlarged portion.

50. An aid as claimed in claim 45, which is provided with a plurality of detachable joint parts that are detachable from the main part to enable fitting of different prostheses to the main part in place of the detached joint part or parts.

51. An aid as claimed in claim 37, wherein the aid comprises a further facsimile skeletal part incorporating a socket part for receiving the joint part or the head of a prosthesis replacing the joint part therein.

52. An orthopaedic hip replacement demonstration aid for demonstrating surgical orthopaedic hip replacement techniques, the aid comprising a facsimile pelvis including a main pelvis part and a detachable socket part for detachable attachment to the main pelvis part by means of the engagement of the socket part in a receiving recess in the main pelvis part, whereby, after a surgical procedure has been carried out on the socket part while attached to the main pelvis part, the socket part may be detached from the main pelvis part and replaced by a fresh socket part.

53. An aid as claimed in claim 52, wherein the socket part is made from a material that allows for milling, reaming, drilling, nibbling or broaching of the socket part to allow for a resurfacing, primary or minimal invasive surgical procedure.

54. An aid as claimed in claim 52, wherein the socket part is detachably attached to the main pelvis part by means of an interference locking fit or a connecting screw.

55. An aid as claimed in claim 52, wherein the socket part is detachably attached to the main pelvis part by means of an arrangement that prevents relative rotation between the joint part and the enlarged portion.

56. An aid as claimed in claim 52 in association with a second aid comprising a facsimile skeletal bone including an elongate main part having an enlarged portion at one end, and a detachable joint part for attachment to the enlarged portion by means of projecting portion of one of the parts that extends into a receiving recess in the other of the parts, whereby, after a surgical procedure has been carried out on the joint part while attached to the main part, the joint part may be detached from the main part and replaced by a fresh joint part.

57. An aid as claimed in claim 56, wherein the detachable socket part and the detachable joint part are interconnected by ligament means.

58. An aid as claimed in claim 57, wherein the ligament means is resiliently expandable to enable the joint part to be manipulated relative to the socket part in such a manner as to mimic dislocation of a skeletal joint within a socket for the purpose of a surgical procedure.

59. An orthopaedic skeletal joint demonstration aid for demonstrating surgical orthopaedic techniques, the aid comprising a first facsimile skeletal bone, a second facsimile skeletal bone, and ligament means connecting the first facsimile skeletal bone to the second facsimile skeletal bone and resiliently expandable to enable the first facsimile skeletal bone to be manipulated relative to the second facsimile skeletal bone in such a manner as to mimic dislocation of a skeletal joint for the purpose of a surgical procedure.

60. An aid as claimed in claim 59, wherein the ligament means comprises sling means for detachable connection to at least one of the facsimile skeletal bones and cord means connecting the sling means to another of the facsimile skeletal bones.

61. An aid as claimed in claim 60, wherein the sling means comprises a first sling part for detachable connection to the first facsimile skeletal bone and a second sling part for detachable connection to the second facsimile skeletal bone.

62. An aid as claimed in claim 60, wherein the sling means comprises at least one length of fabric that is detachably connectable to said at least one facsimile skeletal bone by being wrapped about said one facsimile skeletal bone.

63. An aid as claimed in claim 60, wherein the sling means comprises Velcro strip means for detachable connection of the sling means to said at least one facsimile skeletal bone.

64. An aid as claimed in claim 60, wherein the cord means is connected to the sling means by being passed through a hole in the sling means.

65. An aid as claimed in claim 60, wherein the cord means is connected to the sling means by being knotted or provided with a ferrule at one end.

66. An orthopaedic skeletal joint demonstration aid for demonstrating surgical orthopaedic techniques, the aid comprising a first facsimile skeletal bone, a second facsimile skeletal bone, and ligament means connecting the first facsimile skeletal bone to the second facsimile skeletal bone and detachable from one of the facsimile skeletal bones for the purpose of demonstrating a surgical procedure.

67. An aid as claimed in claim 59, wherein the first facsimile skeletal bone includes a socket part and the second facsimile skeletal bone includes a joint part for engagement with the socket part.

68. An aid as claimed in claim 45, incorporating parts detachably connectable to one another by magnetic attraction.

69. An aid as claimed in claim 45, which is adapted for the attachment of at least one facsimile soft tissue portion thereto.

70. An aid as claimed in claim 45, incorporating an axial passage adapted to detachably receive a shaft of a prosthesis such that a head of the prosthesis projects from the axial passage.

71. An orthopaedic skeletal joint demonstration aid for demonstrating surgical orthopaedic techniques, the aid comprising a first facsimile skeletal bone, a second facsimile skeletal bone, ligament means connecting the first facsimile skeletal bone to the second facsimile skeletal bone, and adjustable connection means connecting the ligament means to at least one of the facsimile skeletal bones and adjustable to vary the length of the ligament means between the first facsimile skeletal bone and the second facsimile skeletal bone.

72. An aid as claimed in claim 71, wherein the adjustable connection means is in the form of spring toggle means normally clamped to the ligament means and compressible against a spring force to enable its position on the ligament means to be adjusted.
